(19) United States
(12) Patent Application Publication
(10) Pub. No.: US 2024/0341337 A1
(43) Pub. Date: Oct. 17, 2024

(54) METHOD FOR CREATING MASKING COMPOSITIONS

(71) Applicant: GIVAUDAN SA, VERNIER (CH)

(72) Inventors: Yuangang ZHANG, Cincinnati, OH (US); Lisa Maria WIJNEN, Naarden (NL); Georgios Andreas KRINTIRAS, Vleuten (NL); Rahul Karthik SIVAGAMINATHAN, Cincinnati, OH (US); Yong Hua XU, Cincinnati, OH (US)

(21) Appl. No.: 18/681,588

(22) PCT Filed: Sep. 1, 2022
(86) PCT No.: PCT/EP2022/074367

§ 371 Date: Feb. 6, 2024

Related U.S. Application Data
(60) Provisional application No. 63/239,629, filed on Sep. 1, 2021.

(51) Int. Cl. A23L 27/00; A23J 3/14; G01N 30/02; G01N 30/88

(52) U.S. Cl. CPC A23L 27/84; A23J 3/14; G01N 30/88; G01N 2030/025; G01N 2030/8831

(57) ABSTRACT

Methods provide for creating compositions for masking non-animal derived protein off-notes. One embodiment of a method includes (a) selecting a non-animal protein for analysis; (b) identifying off-note compounds in the non-animal derived protein; (c) selecting possible antagonist molecules; (d) screening of the antagonist molecules for efficacy of masking the off-note compounds; and (e) combining the efficacious antagonist molecules to form a masking composition. The screening of the antagonist molecules is performed using an aroma blending device configured to deliver at least one aroma substance to the nasal cavities of a user.

### TECHNICAL FIELD

The present disclosure relates systems and methods for off-note masker creation. More particularly, the present disclosure relates to systems and methods for the digitized creation of off-note maskers. Even more particularly, the present disclosure relates to systems and methods for creating maskers for suppressing off-notes of non-animal derived proteins contained in consumables.

### BACKGROUND

The use of non-animal derived proteins in foodstuffs to replace animal raw materials such as egg or milk, but also meat is becoming increasingly important due to the benefits of protein in the diet. While consumers expect their food and beverage products to have multi-functional benefits, consumers still have high expectations that those products deliver great taste along with efficacy in terms of health benefits. Because each type of protein has its own inherent taste, formulating protein into food and beverage products can produce distinctive tastes, i.e off-notes, perceived as unappealing.

An off-note is a flavour note that is not natural or up to the standard owing to deterioration or contamination. For example, the beany and soy flavour note is desirable for soy milk or tofu in Asian food but it becomes an undesirable flavour in meat analogues. The same is true for pea proteins and other non-animal derived proteins. Add in the complexity that for each non-animal protein there are two types of off-notes, i.e. volatile aroma compounds which give off the aroma and non-volatile compounds, that are contributing to the unappealing taste experience of consumers.

Accordingly, when creating maskers there is a need for the ability to digitize the approach to off-note masker creation. By understanding the volatiles and the taste off-notes from non-animal derived proteins, identification and creation of the best masking solution for each specific protein is achievable.

### SUMMARY

In an illustrative embodiment, a method for creating compositions for masking non-animal derived protein off-notes comprises the steps of: (a) selecting a non-animal protein for analysis; (b) identifying off-note compounds in the non-animal derived protein; (c) selecting possible antagonist molecules; (d) screening of the antagonist molecules for efficacy of masking the off-note compounds; and (e) combining the efficacious antagonist molecules to form a masking composition. The screening of the antagonist molecules is performed using an aroma blending device configured to deliver at least one aroma substance to the nasal cavities of a user.

These and other features, aspects and advantages of specific embodiments will become evident to those skilled in the art from a reading of the present disclosure.

### DETAILED DESCRIPTION

The following text sets forth a broad description of numerous different embodiments of the present disclosure. The description is to be construed as exemplary only and does not describe every possible embodiment since describing every possible embodiment would be impractical, if not impossible. It will be understood that any feature, characteristic, component, composition, ingredient, product, step or methodology described herein can be deleted, combined with or substituted for, in whole or part, any other feature, characteristic, component, composition, ingredient, product, step or methodology described herein. Numerous alternative embodiments could be implemented, using either current technology or technology developed after the filing date of this patent, which would still fall within the scope of the claims. All publications and patents cited herein are incorporated herein by reference.

The present disclosure relates to the growing demand for and the number of different consumables that contain non-animal derived proteins. One consequence of this is that the off-notes present in these proteins tend to vary and there is not a “one size fits all” solution to mask these off-notes.

“Non-animal derived protein” refers to protein preparations made from raw materials including, but not limited to, grain (rice, millet, maize, barley, wheat, oat, sorghum, rye, teff, triticale, amaranth, buckwheat, quinoa): legume or pulses, beans (such as soybean, mung beans, faba beans, lima beans, runner beans, kidney beans, navy beans, pinto beans, azuki beans, and the like), peas (such as green peas, yellow peas, chickpeas, pigeon peas, cowpea, and black-eyed peas and the like), sesame, garbanzo, potatoes, lentils, and lupins: seed and oilseed (black mustard, India mustard, rapeseed, canola, safflower, sunflower seed, flax seed, hemp seed, poppy seed, pumpkin, chia, sesame): nuts (almond, walnut, Brazil, Macadamia, cashews, chestnuts, hazelnuts, pine, pecans, peanuts, pistachio and gingko); algal (kelp, wakame, spirulina, chlorella): mycoprotein and/or fungal protein.

The term “off-note” refers to an unpleasant after taste that develops over time after consumption of consumables.

One of the most important criterion for consumer acceptance of foods is flavor. Proteins have little flavor of their own, but influence flavor perception. Protein ingredients both transmit undesirable off-notes to foods and reduce perceived impact of desirable flavorants. In response, Applicants have developed systems and methods for the digitized creation of maskers that makes it possible to provide non-animal derived protein ingredients and products containing non-animal derived proteins with an improved flavor profile with reduced off-notes.

According to the present disclosure, a method for creating compositions for masking non-animal derived protein off-notes may include the following steps, a) selecting a non-animal protein for analysis; b) identifying off-note compounds in the non-animal derived protein; c) selecting possible antagonist molecules; d) screening of the antagonist molecules for efficacy of masking the off-note compounds; and e) combining the efficacious antagonist molecules to form a masking composition. The masking compositions may be a combination of volatile and non-volatile masking compounds. In another example, the masking composition may also include other optional ingredients for particular applications.

The masking composition of the present disclosure may be used in a wide variety of consumables or applications and is not restricted to any particular physical mode or product form. According to the present disclosure, the term “consumable” refers to products for consumption by a subject, typically via the oral cavity (although consumption may occur via non-oral means such as inhalation), for at least one of the purposes of enjoyment, nourishment, or health and wellness benefits. Consumables may be present in any form including, but not limited to, liquids, solids, semi-solids, tablets, capsules, lozenges, strips, powders, gels, gums, pastes, slurries, syrups, aerosols and sprays. The term also refers to, for example, dietary and nutritional supplements. Consumables include compositions that are placed within the oral cavity for a period of time before being discarded but not swallowed. It may be placed in the mouth before being consumed, or it may be held in the mouth for a period of time before being discarded.

Broadly, consumables include, but are not limited to, foodstuffs of all kinds, confectionery products, baked products, sweet products, savoury products, fermented products, dairy products, beverages, oral care products, nutraceuticals and pharmaceuticals.

In certain embodiments, consumables, for example, meat analogs include a high concentration of non-animal derived protein.

For purposes of illustration, the method for creating compositions for masking non-animal derived protein off-notes described herein will be directed to the masking of off-notes associated with pea protein isolates and concentrates, although the methodology is applicable to any non-animal derived protein and off-notes according to the present disclosure.

### Selecting a Non-Animal Protein for Analysis

In accordance with one embodiment, a number of commercially available pea protein isolates and concentrates were selected for analysis. For example, pea protein isolate (Nutralys® F85M, Roquette, Lestrem, France) or (PisaneR M9, Coscura, Belgium) were selected among others.

### Identifying Off-Note Molecules in the Non-Animal Derived Protein

In one embodiment, the pea protein isolates were then analyzed by gas chromatography-olfactometry (GC-O) technique in order to identify off-note volatiles. The GC-O technique couples traditional gas chromatographic analysis with sensory detection in order to study complex mixtures of odorous substances and to identify odor active compounds. In one embodiment, off-note volatiles present in pea protein included, for example, propyl-iso-2 methoxy-3-pyrazine (pea-like), isobutyl-3-methoxy-2-pyrazine (earthy green), heptenal-2-trans (cardboardy), heptenal-4-cis (vegetal) and hexanol (soapy).

### Selection of Antagonist Molecules

Antagonism in chemistry refers to the involvement of multiple agents which reduces their overall effect, and the aroma antagonism between odorants at the receptor level was demonstrated by Oka, et al in 2004 (Oka, Y. et al. Chem. Senses, 29:815-822, 2004). However, it has not been applied in masking flavour creations. The criteria that may be used to pre-select molecules for antagonist screening are (1) Avoiding the same class of molecules as off-note odorants, because the same family or analogue molecules tend to work together to enhance each other; and (2) Selecting molecules or natural extracts that are congruent to savoury flavours such as garlic oil, onion extract and molasses distillate, which are commonly used as savoury flavouring ingredients.

In accordance with the present disclosure, antagonist molecules may be used alone or in combination in order to create compositions suitable for masking or modifying the undesirable off-note(s) in a particular non-animal derived protein. Historically, masking undesirable off-notes in foods or beverages involved using more sugar or fat to cover bitterness and adjust flavor perception. Flavorists simply “over flavored” their products to hide the offending taste. These traditional methods are wholly unsatisfactory, especially for health-conscious consumers where reduced fat and sugar content is a common goal.

Various non-animal proteins provide undesirable off-notes. Particularly, undesirable off-notes are the beany, bitter, grassy, astringent, earthy, chalky, and rancid off-notes from pea and soy proteins. The addition of antagonist molecules may block, mask or modify the off-notes and make them less apparent or unnoticeable. Non-animal proteins will thereby lose their beany/bitter/grassy/astringent/earthy/chalky/rancid taste.

According to one embodiment, suitable antagonist molecules for use in accordance with the present disclosure include fatty acids including, but not limited to, nonanoic acid, decanoic acid, dodecanoic acid, tetradecanoic acid, hexadecanoic acid, oleic acid, octanoic acid, 9-decenoic and hexanoic acid.

In another embodiment, suitable antagonist molecules include carbonyls including, but not limited to, acetone, acetyl propionyl. 2-heptanone, 2-nonanone, 2-undecanone and cis-4-heptenal. In another embodiment, suitable antagonist molecules include sulfur, including, but not limited to, isothiocyanates, methyl sulfide, diallyl disulfide, propenyl disulfide, dimethyl sulfide, dimethyl trisulfide and extracts of alliaceous ingredients. In another embodiment, the sulfur components may be found in sulfur containing oils such as, for example, garlic oil, onion oil, mustard oil and horseradish oil.

In another embodiment, suitable antagonist molecules include sweet browns including, but not limited to, maltol, vanillin, cyclopentenolone, furaneol, vanilla extracts, vanilla derivatives, caramel extracts and condensed milk derivatives.

In another embodiment, suitable antagonist molecules include esters including, but not limited to, ethyl cyclohexanoate, ethyl succinate, ethyl lactate, ethyl caprate, ethyl dodecanoate, ethyl myristate, ethyl palmitate and ethyl oleate. In another embodiment, suitable masking agents include sweeteners including but not limited to, steviol glycosides such as rebaudiosides; rebusodide, swingle extract, mogroside V, erythritol, glucosylated steviol glycosides, honey distillates and sugar distillates.

In another embodiment, suitable antagonist molecules include lactones including, but not limited to, gamma decalactone, delta decalactone, delta dodecalactone, gamma undecalactone and massoia lactone. In another embodiment, masking agents include juice derivatives including, but not limited to, strawberry, cucumber, apple, cherry, kiwi and apricot.

According to one embodiment, suitable antagonist molecules for use in accordance with the present disclosure include terpenes including, but not limited to, fenchol, terpineol, caryophyllene, bisabolene, farnoscene and farnesol. In another embodiment, suitable terpenes include, but are not limited to, carotenes (such as, for example, alpha-carotene, beta-carotene, gamma-carotene, delta-carotene, lycopene, neurosporene, phytofluene, phytoene), and xanthophylls (such as, for example, canthaxanthin, cryptoxanthin, aeaxanthin, astaxanthin, lutein, rubixanthin): monoterpenes (such as, for example, limonene, perillyl alcohol): sesquiterpenes (such as, for example, caryophyllene, β-caryophyllene, zingiberene): saponins: lipids including: phytosterols, campesterol, beta sitosterol, gamma sitosterol, stigmasterol), tocopherols (vitamin E), and omega-3, -6, and -9) fatty acids (such as, for example, gamma-linolenic acid); triterpenoids (such as, for example, oleanolic acid, ursolic acid, betulinic acid, moronic acid); alpha-pinenes, cis-beta-ocimenes and bisabolenes (such as alpha-bisabolene and gamma-bisabolene.

### Screening of Antagonist Molecules

According to the present disclosure, the screening of antagonist molecules is done utilizing an aroma blending device.

Turning to FIG. 1, in accordance with one embodiment, the aroma blending device 10 may include the following elements (not all elements are shown): a source of carrier gas flow: a regulating means which receives the carrier gas flow and regulates its passage through a plurality of channels; downstream of the regulating means, a plurality of aroma substance containing cartridges 20, one being associated with each channel; and a disseminating means configured to deliver at least one aroma substance to the nasal cavities of a consumer. In one embodiment, the aroma blending device 10 may be an olfactometer device (an instrument capable of exactly dosing a portion of a gaseous phase which is present due to the vapor pressure of a sample or in the form of a liquid or a solid) for example the Virtual Aroma Synthesizer® devices (VAS-Air® or MiniVAS™), both developed and owned by Givaudan. For purposes of the present disclosure, the aroma substance may be volatile off-note molecules or antagonist candidate molecules.

The source of carrier gas may be any suitable source of gas. For example, the carrier gas may be air or nitrogen, and the source may be a compressor. Alternatively, the source may be a pressurized cylinder of gas. The gas flow is conveyed to a regulating means. The regulating means is typically a device that comprises a plurality of channels configured to convey the gas to cartridges 20 containing aroma substance, one cartridge per channel. Thus, by opening and closing channels and maintaining these openings and closings for predetermined times, the regulating means can determine which aroma substances and how much thereof are conveyed and when, and thus alter the nature of the aroma perceived by a user.

The operation of the regulating means may be remote, for example, by computer either wired to the regulating device or operating it wirelessly. In one embodiment, the aroma blending device 10 includes a short-range communication reader for short range communication. Non-limiting examples of short-range communication include. Bluetooth, radio frequency identification (RFID), infrared data association (IrDA), ultra-wideband (TJWB), ZigBee, or any number of other wireless communication protocols or interfaces. In one embodiment, the aroma blending device includes a near-field communication (“NFC”) interface. The NFC interface may have a range of approximately about 4 inches to about 8 inches. The close-range communication with the NFC interface may take place via magnetic field induction 30, allowing the NFC interface to communicate with other NFC device(s) or to retrieve information from an NFC sticker(s) 25 located on the plurality of cartridges 20 as explained in more detail below:

In accordance with another embodiment, the aroma blending device 10 may have a display. Display may function as a touch screen through which a user may interact with the aroma blending device 10.

The cartridges 20 of aroma substance from which the desired aroma is created may be any suitable cartridge capable of containing the desired aroma substances and releasing them when impinged upon by a carrier gas. One particular type is a cartridge 20 comprising a reservoir containing an aroma substance and being provided with inlet and outlet channels to permit ingress of carrier gas into the reservoir and egress of aroma substance-containing carrier gas from the reservoir, the channels being defined by capillaries having internal diameter and length dimensions sufficient to act as closure means to prevent, or substantially prevent, leakage of aroma substance from the reservoir into a head space external of the cartridge when carrier gas flow is interrupted. Such a cartridge is described in U.S. Pat. No. 7,601,297, incorporated herein by reference in its entirety. It has the advantages that it can be small and very slim (about credit card-sized) and that it does not require valve arrangements, merely a supply of carrier gas to entrain the substance from the cartridge and means of directing and regulating this. Such cartridges are easily replaced when empty. FIG. 2a shows a credit-card design 100 for a support body in perspective containing a plurality of cartridges 20 or rack. In another embodiment. FIG. 2b shows a generally cylindrical support body 110 containing a plurality of cartridges 20 or rack. In one embodiment, a rack consists of any number of individual cartridges including, for example, 3, 4, 5, 6, 7, 8, 9, 10, 11, 12, 13, 14, 15 or more cartridges. In some embodiments, the rack of cartridges includes at least five individual cartridges: in another embodiment at least ten individual cartridges: in another embodiment at least fifteen individual cartridges. In another embodiment, the aroma blending device may include more than 1 rack of cartridges, for example at least 2 or 3 racks of cartridges, for the possibility of a total of 30 or 45 individual cartridges loaded into the aroma blending device 10.

The aroma substances that are entrained by the gas flow from the cartridges are conveyed by the carrier to a disseminating means via a conduit. The nature of the disseminating means may be any disseminating means capable of delivering a vapour-phase substance to the nasal cavities of an individual. For example, it may be a small nose piece able to be worn, or it may be a sniffing port connected to the aroma blending device.

In one embodiment, the screening step is carried out using the above described aroma blending device as follows: each off-note molecule(s) identified from a target non-animal derived protein may be loaded into its own individual cartridge or channel (for example, propyl-iso-2-methoxy-3-pyrazine may be loaded into cartridge/channel #1, and isobutyl-3-methoxy-2-pyrazine may be loaded into cartridge/channel #2, etc.); next, the target non-animal protein is diluted as will be discussed below and may be loaded into its own individual cartridge/channel, for example, cartridge/channel #6 to act as the control or base; next, each antagonist candidate molecule that has been identified may be loaded into its own individual cartridge/channel (for example, molasses distillate may be loaded into cartridge/channel #7 and bisabolene may be loaded into cartridge/channel #8, etc). Next, the gas flow on the off-note molecule containing cartridge/channel is first opened, followed by one of the antagonist candidate molecule containing cartridges/channels. The intensity of the flow of the antagonist candidate molecule containing cartridge/channel is adjusted until the off-note is canceled or neutralized by the antagonist candidate molecule, i.e neutralization concentration. This process is repeated for each antagonist candidate molecule containing cartridges/channels so that all of the potential antagonist candidate molecules are screened.

### Combining the Efficacious Antagonist Molecules to Form a Masking Composition

After screening of the potential antagonist candidate molecules is completed, the most effective antagonist molecules are noted. The most effective antagonist molecules are combined together at their neutralization concentration in order to create a masking composition. This masking composition may then be validated by the aroma blending device by evaluating the combined masker cartridge/channel flow (combination of the cartridges/channels containing the most effective antagonist molecules) and the off-note channel flow from a cartridge/channel containing the target non-animal derived protein, for example, a pea protein. According to certain embodiments, the masking composition comprises one or more chemical compounds that are used to solubilize the ingredients. Without limitation, suitable solubilizers include isopropyl alcohol and 1,3 propanediol.

### Uses

The masking composition obtained by and/or obtainable by the methods described herein may, for example, be added to consumables/food products (e.g. as part of a flavour composition) to improve mouthfeel and/or reduce/mask off-notes of the consumable.

In general terms, “mouthfeel” refers to the complexity of perceptions experienced in the mouth as influenced by the aroma, taste, and texture qualities of food and beverage products. From a technical perspective, however, mouthfeel sensations are specifically associated with physical (e.g. tactile, temperature) and/or chemical (e.g. pain) characteristics perceived in the mouth via the trigeminal nerve. Accordingly, they are a consequence of oral-tactile stimulations and involve mechanical, pain and temperature receptors located in the oral mucosa, lips, tongue, cheeks, palate and throat.

Mouthfeel perceptions include, for example, one or more of texture-astringent, burning, cold, tingling, thick, biting, fatty, oily, slimy, foamy, melting, sandy, chalky, watery, acidic, lingering, metallic, body, body sweet, carbonation, cooling, warming, hot, juicy, mouth drying, numbing, pungent, salivating, spongy, sticky, fullness, cohesiveness, density, fracturability, graininess, grittiness, gumminess, hardness, heaviness, moisture absorption, moisture release, mouthwatering, mouthcoating, roughness, slipperiness, smoothness, uniformity, uniformity of bite, uniformity of chew, viscosity, fast-diffusion, full body, salivation and retention.

By “improvement of mouthfeel” it is meant that any one or more of desired mouthfeel perceptions is/are enhanced and/or that any one or more undesirable mouthfeel perceptions is/are reduced.

By “masking of off-notes” it is meant that the intensity and/or length of perception of undesirable attributes in a food product is reduced, as analysed by trained panelists when comparing food comprising an ingredient with off-note masking to food without an added off-note masking ingredient.

According to other embodiments, the disclosed method may be used to reduce or eliminate off-notes imparted by non-animal derived protein such as plant protein. Exemplary plant proteins include soy protein and pea protein. As used herein, soy includes all consumables containing soy in any form, including soy bean oil used either alone, in combination, for example as a nutraceutical, or as a medicament, soy bean curd, soy milk, soy butter or soy paste. The plant protein may comprise algae (such as spirulina), beans (such as black beans, canelli beans, kidney beans, lentil beans, lima beans, pinto beans, soy beans, white beans), broccoli, edamame, nuts (such as almonds, brazil nuts, cashews, peanuts, pecans, hazelnuts, pine nuts, walnuts), peas (such as black eyed peas, chickpeas, green peas), potatoes, oatmeal, seeds (such as chia, flax, hemp, pumpkin, sesame, sunflower), grain (rice, millet, maize, barley, wheat, oat, sorghum, rye, teff, triticale, amaranth, buckwheat, quinoa), seitan (i.e., wheat gluten-based), tempeh, tofu, mycoprotein or fungal protein; insects and leaf protein and mixtures thereof.

According to other embodiments, the disclosed masking composition may be used to reduce or eliminate off-notes imparted by meat analog products containing non-animal protein. “Meat analog” is a food product that approximates the aesthetic qualities and/or chemical characteristics of certain types of meat. The term Meat analogue includes those prepared with textured vegetable proteins (TVP), high moisture meat analogue (HMMA) and low moisture meat analogue (LMMA) products.

### BRIEF DESCRIPTION OF THE DRAWINGS

FIG. 1 is a schematic representation of an aroma blending device including an NFC interface communicating with another NFC device containing a plurality of cartridges via magnetic field induction.

FIG. 2A is a perspective view of an illustrative embodiment of a support body configured in the shape of a credit card containing a plurality of cartridges within the support body.

FIG. 2B is a perspective view of an illustrative embodiment wherein a generally cylindrical support body contains a plurality of cartridges within the support body.

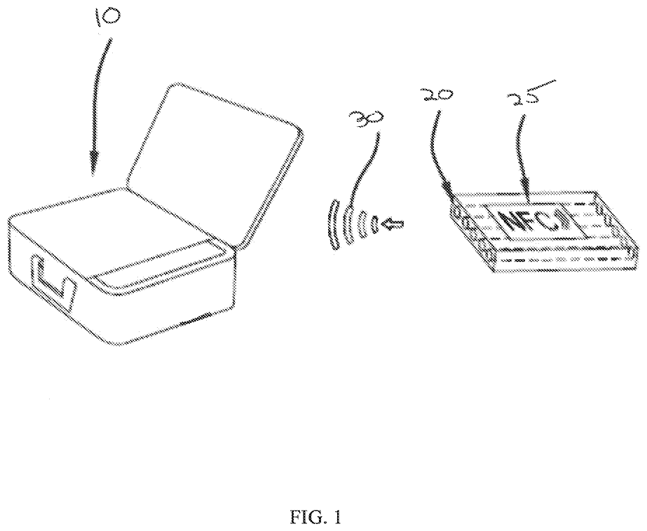

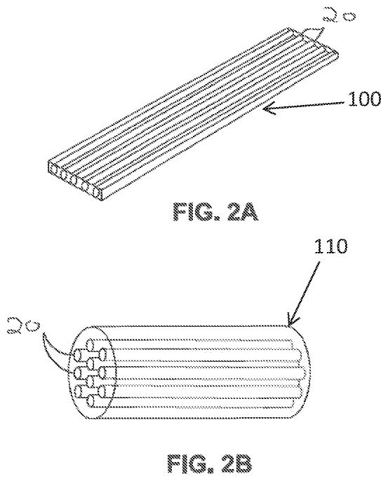

### Example

The following example is given solely for the purpose of illustration and is not to be construed as limitations of the present invention, as many variations of the invention are possible without departing from the spirit and scope of the present disclosure. Using the above-described method, the following exemplary implementation of the method may be performed on a non-animal derived pea protein isolate using a Virtual Aroma Synthesizer® (VAS) aroma blending device.

Pea protein isolate (Pisane® C9, Coscura, Belgium) was selected as the non-animal derived protein for this example. GC-O analysis was performed on the pea protein isolate in order to identify the key off-notes associated with pea protein isolates and concentrates, as shown in Table 1 below.

### Table 1

### Pea Protein Off-Notes for Antagonist Screen

TABLE 1
Pea Protein Off-Notes for Antagonist Screen

| Off-notes | Descriptors |
|---|---|
| propyl-iso-2 methoxy-3-pyrazine (Ex. A) | Pea-like |
| isobutyl-3-methoxy-2-pyrazine (Ex. B) | Earthy green |
| heptenal-2-trans (Ex. C) | Cardboardy |
| heptenal-4-cis (Ex. D) | Vegetal |
| hexanol (Ex. E) | Soapy |

Selection and screening of the antagonist molecules was performed using the VAS aroma blending device. As described above, each off-note molecule from Table 1 was loaded into its own individual cartridge or channel as shown in Table 2 (#'s 1-5). Each off-note molecule was diluted with medium-chain triglyceride (“MCT”) prior to loading into the cartridge/channel. Next, the target non-animal protein was diluted at 2% plus 0.1% xanthan gum in water with a small amount of antifoam agent (0.01%) and was placed into a cartridge/channel (#21). Each antagonist candidate molecule was then diluted with MCT in a pre-determined concentration and was loaded into a cartridge/channel as shown in Table 2 (#'s 6-20). Next, the gas flow (between 0 to 500 ml/min) on the off-note molecule containing cartridge/channel is first opened, followed by one of the antagonist candidate molecule containing cartridges/channels. The intensity of the flow of the antagonist candidate molecule containing cartridge/channel is adjusted until the off-note is canceled or neutralized by the antagonist candidate molecule, i.e neutralization concentration. This process is repeated for each antagonist candidate molecule containing cartridges/channels so that all of the potential antagonist candidate molecules are screened.

### Table 2

### Antagonist Screening for Pea Protein Off-Notes

TABLE 2
Antagonist Screening for Pea Protein Off-Notes

| | | | Off-note | Off-note | Off-note | Off-note | Off-note |
|---|---|---|---|---|---|---|---|
| | | Channel | Ex. A | Ex. B | Ex. C | Ex. D | Ex. E |
| Channel | Antagonist | (Conc. %) | Flow Rate | Flow Rate | Flow Rate | Flow Rate | Flow Rate |
| 1 | NA | 0.01 | 300 | | | | |
| 2 | NA | 0.001 | | 150 | | | |
| 3 | NA | 0.01 | | | 185 | | |
| 4 | NA | 0.10 | | | | 15 | |
| 5 | NA | 1.0 | | | | | 150 |
| 21* | NA | 2.0 | | | | | |
| 6 | Caryophyllene | 1.0 | 100+++ | + | − | 150++ | 200-225+ |
| 7 | Ethyl lactate | 1.0 | 90+++ | 100+++ | 200++ | 200++ | −− |
| 8 | Molasses | Neat | 50++ | − | 65+++ | − | 150++ |
| | distillate | | | | | | |
| 9 | Ethyl oleate | Neat | −− | − | − | 240+ | −− |
| | rich | | | | | | |
| 10 | Chamomile | 10% PG | | | 150+++ | | 150++ |
| 11 | Coffee Extract | 10% PG/ | 90++ | 50++ | 70++ | | 200++ |
| | Green | water | | | | | |
| 12 | Garlic Oil | 0.01 | 25+ | 80+ | 20++ | 15+ | 150++ |
| 13 | Mustard Oil | 0.10 | 400− | − | − | 325++ | − |
| 14 | Onion Oil | 0.01 | 30++ | 20-25+ | 55+ | 25++/− | 60++ |
| 15 | Chamomile | Neat | 55+++ | 40++/− | 75-100+/− | 90++ | − |
| 16 | Bisabolene | Neat | − | − | − | − | 150+ |
| 17 | Ethyl | 20.00 | − | 100+++/− | 35++ | − | 300+ |
| | Palmitate | | | | | | |
| 18 | Ally1 | 10.00 | 165+/− | − | 100+++ | − | 175++ |
| | Cyclohexyl | | | | | | |
| | Proprionate | | | | | | |
| 19 | Esterly | 0.0010% | 200+++ | 50+++ | 150++ | 400++ | −− |
| | | | | | 225+++ | | |
| 20 | Diethyl | Neat | 250++ | 300++ | 200+/− | 200++ | −− |
| | Succinate | | | | | | |

*(2% Pisane C9 + 0.1% Xanthan gum)

* (2% Pisane C9+0.1% Xanthan gum)

As shown in Table 2, the masking effect was marked by + or − signs while +++ being most effective, ++ in the middle, and + some effect, +/− being in between, and − being no effect. The numeric number in the column refers the flow rate on VAS aroma blending device. Based on the results of the screening, the most effective antagonist molecules are combined together at their neutralization concentration in order to create a masking composition. For example, a masking composition in accordance with the present disclosure is shown in Table 3.

### Table 3

### Pea Masking Composition

TABLE 3
Pea Masking Composition

| Ingredients | (wt. %) |
|---|---|
| Molasses distillate | 50.00 |
| Pea peptide | 10.00 |
| Isopropyl alcohol | 50.00 |
| 1,3 Propanediol | 300.00 |
| Diethyl succinate | 0.38 |
| Ethyl lactate | 0.44 |
| Caryophyllene | 0.44 |
| Garlic oil | 0.06 |
| Onion oil | 0.01 |
| Water | 588.67 |

This masking composition may then be validated by the aroma blending device by evaluating the combined masker cartridge/channel flow (combination of the cartridges/channels containing the most effective antagonist molecules) and the off-note channel flow from a cartridge/channel (#21) containing the target non-animal derived protein, for example, a pea protein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method for creating compositions for masking non-animal derived protein off-notes, the method comprising:
a) selecting a non-animal protein for analysis;
b) identifying off-note compounds in the non-animal derived protein;
c) selecting possible antagonist molecules;
d) screening the antagonist molecules for efficacy of masking the identified off-note compounds; and
e) combining the efficacious antagonist molecules to form a masking composition,
wherein the screening of the antagonist molecules is performed using an aroma blending device configured to deliver at least one aroma substance to the nasal cavities of a user.

2. The method according to claim 1, wherein the aroma blending device comprises:
a) a source of carrier gas;
b) a regulating means which receives the carrier gas and regulates the passage of the carrier gas through a plurality of channels;
c) a plurality of aroma substance containing cartridges in fluid communication with the plurality of channels;
d) a disseminating means configured to deliver the at least one aroma substance to the nasal cavities of the user.

3. The method according to claim 1, wherein the aroma blending device is an olfactometer device.

4. The method according to claim 1, wherein the carrier gas comprises air, nitrogen, or combinations thereof.

5. The method according to claim 2, wherein the regulating means is a device that comprises a plurality of channels configured to convey the carrier gas to the plurality of aroma substance containing cartridges, wherein there is one cartridge per channel.

6. The method according to claim 5, wherein the regulating means is configured to determine which aroma substances and how much thereof are conveyed and when, thereby altering the nature of the aroma perceived by the user.

7. The method according to claim 1, wherein the antagonist molecules are selected from the group consisting of caryophyllene, ethyl lactate, molasses distillate, ethyl oleate, chamomile, green coffee extract, garlic oil, mustard oil, onion oil, bisabolene, ethyl palmitate, allyl cyclohexyl proprionate, diethyl succinate, and combinations thereof.

8. The method according to claim 1, wherein the aroma blending device comprises a near-field communication (NFC) interface.

9. The method according to claim 1, wherein the non-animal derived protein comprises a pea protein.

10. The method according to claim 9, wherein the pea protein imparts one or more off-notes selected from the group consisting of propyl-iso-2 methoxy-3-pyrazine, isobutyl-3-methoxy-2-pyrazine, heptenal-2-trans, heptenal-4-cis, hexanol, and combinations thereof.

11. The method of claim 2, wherein the screening step (d) comprises:
(d)(1) loading an off-note compound from the selected a non-animal protein into its own individual cartridge or channel of the aroma blending device;
(d)(2) loading a dilution of the selected the non-animal protein into its own individual cartridge or channel of the aroma blending device as a control;
(d)(3) loading a candidate antagonist molecule into its own individual cartridge or channel of the aroma blending device;
(d)(4) opening the gas flow of the cartridge or channel of the aroma blending device containing the off-note compound from the selected a non-animal protein;
(d)(5) opening the gas flow of the cartridge or channel of the aroma blending device containing the candidate antagonist molecule; and
(d)(6) adjusting the intensity of the flow of the candidate antagonist molecule containing cartridge or channel until the off-note is neutralized.

12. The method of claim 1, wherein suitable antagonist molecules comprise fatty acids selected from the group consisting of nonanoic acid, decanoic acid, dodecanoic acid, tetradecanoic acid, hexadecanoic acid, oleic acid, octanoic acid, 9-decenoic, hexanoic acid, and combinations thereof.

13. The method of claim 1, wherein suitable antagonist molecules comprise carbonyls selected from the group consisting of acetone, acetyl propionyl, 2-heptanone, 2-nonanone, 2-undecanone, cis-4-heptenal, and combinations thereof.

14. The method of claim 1, wherein suitable antagonist molecules comprise sulfur containing compounds selected from the group consisting of isothiocyanates, methyl sulfide, diallyl disulfide, propenyl disulfide, dimethyl sulfide, dimethyl trisulfide, extracts of alliaceous ingredients, and combinations thereof.

15. The method of claim 1, wherein suitable antagonist molecules comprise sweet browns selected from the group consisting of maltol, vanillin, cyclopentenolone, furaneol, vanilla extracts, vanilla derivatives, caramel extracts, condensed milk derivatives, and combinations thereof.

16. The method of claim 1, wherein suitable antagonist molecules comprise esters selected from the group consisting of ethyl cyclohexanoate, ethyl succinate, ethyl lactate, ethyl caprate, ethyl dodecanoate, ethyl myristate, ethyl palmitate and ethyl oleate. In another embodiment, suitable masking agents include sweeteners including but not limited to, steviol glycosides such as rebaudiosides; rebusodide, swingle extract, mogroside V, erythritol, glucosylated steviol glycosides, honey distillates, sugar distillates, and combinations thereof.

17. The method of claim 1, wherein suitable antagonist molecules comprise lactones selected from the group consisting of gamma decalactone, delta decalactone, delta dodecalactone, gamma undecalactone, massoia lactone, and combinations thereof.

18. The method of claim 1, wherein suitable antagonist molecules comprise terpenes selected from the group consisting of alpha-pinenes, bisabolenes, caryophyllene, carotenes, cis-beta-ocimenes fenchol, monoterpenes, terpincol, triterpenoids, farnoscene, farnesol, sesquiterpenes, xanthophylls, and combinations thereof.
